SEQUENCE LISTING (17 sequences)

SEQ ID NO: 1 - DNA, 6 bp, Artificial Sequence
  Description of Artificial Sequence Synthetic oligonucleotide
  modified_base (1): Fl-t
tgactc

SEQ ID NO: 2 - DNA, 28 bp, Artificial Sequence
  Description of Artificial Sequence Synthetic oligonucleotide
gctcacgcatgtgagcgtgagcgagtca

SEQ ID NO: 3 - DNA, 40 bp, Artificial Sequence
  Description of Artificial Sequence Synthetic oligonucleotide
gctacctgtcacgcatgtgagcgtgacaggtagcgagtca

SEQ ID NO: 4 - DNA, 12 bp, Artificial Sequence
  Description of Artificial Sequence Synthetic oligonucleotide
tgaacttagctc

SEQ ID NO: 5 - DNA, 12 bp, Artificial Sequence
  Description of Artificial Sequence Synthetic oligonucleotide
  modified_base (1): H2N-t
tgaacttagctc

SEQ ID NO: 6 - DNA, 12 bp, Artificial Sequence
  Description of Artificial Sequence Synthetic oligonucleotide
  modified_base (1): Fl-t
tgaacttagctc

SEQ ID NO: 7 - DNA, 34 bp, Artificial Sequence
  Description of Artificial Sequence Synthetic oligonucleotide
gctcacgcatgtgagcgtgagcgagctaagttca

SEQ ID NO: 8 - DNA, 46 bp, Artificial Sequence
  Description of Artificial Sequence Synthetic oligonucleotide
gctacctgtcacgcatgtgagcgtgacaggtagcgagctaagttca

SEQ ID NO: 9 - DNA, 46 bp, Artificial Sequence; identical to SEQ ID NO: 8
  Description of Artificial Sequence Synthetic oligonucleotide
  modified_base (46): a-(disulfide label)

SEQ ID NO: 10 - DNA, 62 bp, Artificial Sequence
  Description of Artificial Sequence Synthetic oligonucleotide
  modified_base (1): Fl-t
  modified_base (62): c-biotin
tgaacttagctcgctacctgtcacgcatgtgagcgtgacaggtagcgagctaagttcacaac

SEQ ID NO: 11 - DNA, 50 bp, Artificial Sequence
  Description of Artificial Sequence Synthetic oligonucleotide
  modified_base (50): c-biotin
gctacctgtcacgcatgtgagcgtgacaggtagcgagctaagttcacaac

SEQ ID NO: 12 - DNA, 72 bp, Artificial Sequence
  Description of Artificial Sequence Synthetic oligonucleotide
  modified_base (1): Fl-t
  modified_base (72): c-biotin
tgaacttagctcgctgaggtacctgtcacgcatgtgagcgtgacaggtacctcagcgagctaagttcacaac

SEQ ID NO: 13 - DNA, 58 bp, Artificial Sequence
  Description of Artificial Sequence Synthetic oligonucleotide
  modified_base (1): phosphate-t
  modified_base (58): c-biotin
tgaggtacctgtcacgcatgtgagcgtgacaggtacctcagcgagctaagttcacaac

SEQ ID NO: 14 - DNA, 14 bp, Artificial Sequence
  Description of Artificial Sequence Synthetic oligonucleotide
  modified_base (1): Fl-t
tgaacttagctcgc

SEQ ID NO: 15 - DNA, 72 bp, Artificial Sequence
  Description of Artificial Sequence Synthetic oligonucleotide
  modified_base (1): Fl-t
  modified_base (72): c-biotin
tgaacttagctcgctgaggtacctgtcacgcagtgtagcgtgacaggtacctcagcgagctaagttcacaac

SEQ ID NO: 16 - DNA, 10 bp, Artificial Sequence
  Description of Artificial Sequence Synthetic oligonucleotide
  modified_base (6)..(10): a, c, g, t, unknown or other
gagtcnnnnn

SEQ ID NO: 17 - DNA, 10 bp, Artificial Sequence
  Description of Artificial Sequence Synthetic oligonucleotide
  modified_base (6)..(10): a, c, g, t, unknown or other
ggatcnnnnn

(19) United States
(12) Patent Application Publication
(10) Pub. No.: US 2007/0009944 A1
(43) Pub. Date: Jan. 11, 2007

(54) Assay for nucleic acid ligase and nucleic acid nuclease

(75) Inventors: Richard Bowater, Norwich (GB); Julea Nicole Butt, Norwich (GB); Benjamin Oliver Seager Scott, Norwich (GB)

Correspondence Address: HELLER EHRMAN WHITE & MCAULIFFE LLP, 1717 RHODE ISLAND AVE, NW, WASHINGTON, DC, 20036-3001, US

(73) Assignee: University of East Anglia, Norwich (GB)

(21) Appl. No.: 11/474,956

(22) Filed: Jun. 27, 2006

Related U.S. Application Data
(60) Provisional application No. 60/695,538, filed on Jul. 1, 2005.
(60) Provisional application No. 60/750,807, filed on Dec. 16, 2005.

(51) Int. Cl. C12Q 1/68; C07H 21/04

(52) U.S. Cl. 435/6; 536/24.3

(57) ABSTRACT

A method of determining activity of a nucleic acid ligase or a nucleic acid nuclease is described. This method comprises the steps of: (i) providing a nucleic acid molecule comprising a hairpin with a single-stranded loop and a double-stranded stem containing a target site for the nucleic acid ligase and/or the nucleic acid nuclease, wherein the nucleic acid molecule has a first end tethered to a surface and a second end remote from the first end, and wherein a detectable label is attached to the nucleic acid molecule either at the second end or between the target site and the second end; (ii) contacting the nucleic acid molecule with the nucleic acid ligase or the nucleic acid nuclease; and (iii) detecting the presence or absence of the detectable label, thereby determining activity of the nucleic acid ligase or the nucleic acid nuclease.

### BACKGROUND OF THE INVENTION

1. Field of the Invention

The present invention relates to methods of determining the activity of, or detecting, enzymes such as nucleic acid ligases and nucleic acid nucleases. The invention also includes a nucleic acid molecule suitable for use in such methods.

2. Description of the Prior Art

Ligases seal breaks (or “nicks”) in the backbone of duplex DNA, RNA or DNA/RNA hybrids and also in ssRNA. Thus, these enzymes are essential to all organisms. Nucleases act in opposite fashion by cleaving strands in the backbone of duplex DNA, RNA or DNA/RNA hybrids, thereby creating single-strand nicks (or double-strand breaks) in the nucleic acid. Eukaryotic DNA ligases use ATP as a cofactor, whereas essential eubacterial DNA ligases use NAD⁺. Based on this different cofactor specificity, NAD⁺-dependent DNA ligases have been suggested as a promising target for broad-spectrum antibacterial compounds.

Denaturing gel electrophoresis is currently the most common method used to assay activity of enzymes such as ligases and nucleases. This method involves migration of samples down the gel in response to an electric current, with strands of DNA separating out based on size, as shorter strands transit faster than longer strands. These strands are then detected either through reporter molecules (traditionally radiolabels, although fluorophores are becoming increasingly prevalent) or staining. The denaturing gel electrophoresis method is time-consuming, with gel preparation, electrophoresis and autoradiography or imaging taking several hours. The process is also labour-intensive and allows a limited sample number to be screened per gel. If, for example, DNA ligases are to be exploited as a drug target and in molecular biology experiments, a high-throughput assay system would be preferred.

The ability to produce nucleic acids of defined sequence by chemical synthesis has revolutionised analysis in the biological, pharmaceutical and forensic arenas. Synthetic oligonucleotides have become essential elements of methods designed for sequence specific DNA detection and the characterisation of DNA interactions with proteins, drugs and other chemicals. The selectivity and specificity of these approaches is in large part due to the inherent chemical properties of DNA. Many of these properties are evident in the operation of “molecular beacons”, pre-eminent amongst DNA-containing biosensors, which exploit DNA hybridisation chemistry (see Broude, 2002, Trends Biotechnol. 20: 249-256; Heyduk & Heyduk, 2002, Nature Biotechnol. 20: 126-127; WO03/078449 [Heyduk]; and WO03/064657 [Heyduk]). Molecular beacons are DNA hairpins terminated at opposite ends by a fluorophore and quencher. The presence of a complementary DNA sequence opens the hairpin, separates the fluorophore and quencher and a signal is generated.

The majority of assays exploiting DNA hairpins have been developed for homogeneous detection of complementary DNA sequence through changes in fluorescence intensity. However, opportunities to exploit hairpins for the detection of a wider range of analytical targets with alternate methods of signal transduction have begun to be realised. Hairpins have been designed that report quantitatively on the activity of DNA processing enzymes (Heyduk & Heyduk, 2002, supra; Tang et al., 2003, Nucleic Acids Res. 31: e148). Heterogeneous assays in which hairpins are tethered to surfaces that form an integral part of the signal transduction method are also known (Du et al., 2003, J. Am. Chem. Soc. 125: 4012-4013). An example of this approach developed by Fan et al. (2003, Proc. Nat. Acad. Sci. USA 100: 9134-9137) utilises an electroactive ferrocene-tagged DNA hairpin that self-assembles onto a gold electrode by gold-thiol chemistry. In the absence of a target (i.e. a complementary DNA molecule), the hairpin structure holds the ferrocene tag into close proximity with the electrode surface, thus ensuring rapid electron transfer and efficient redox of the ferrocene label. However, on hybridisation with a target sequence, a large change in redox currents is observed, presumably because the ferrocene label is separated from the electrode surface. The method thus allows sequence-specific detection of DNA.

In Liu et al. (2005, Analyst 130: 350-357), a molecular beacon has been used in an assay to monitor activity of Escherichia coli DNA ligase. The molecular beacon, comprising a DNA hairpin with a fluorophore and a quencher linked to the 5′- and 3′-ends of the hairpin stem, respectively, is hybridised with two single-stranded DNA segments which form a hybrid with a nick. Ligation of the two single-stranded DNA segments, i.e. repair of the nick by the DNA ligase, causes the molecular beacon stem to be forced apart, leading to separation of the fluorophore and quencher, thereby enhancing fluorescence signal.

Despite the advances in assay systems for measuring nucleic acid ligase and/or nucleic acid nuclease systems, there remains the need for improving the assays to simplify the procedure and render them more cost-effective.

### SUMMARY OF THE INVENTION

It is an object of the invention to provide an improved method to detect and/or quantify nucleic acid ligase and/or nucleic acid nuclease activity. It is a further object of the invention to provide components suitable for use in the improved method.

The present inventors have developed a method which allows high throughput detection of nucleic acid ligases or nucleic acid nucleases. The method is highly sensitive and can monitor a broad range of enzyme activity.

According to a first aspect of the invention there is provided a method of determining activity of a nucleic acid ligase or a nucleic acid nuclease, the method comprising the steps of:
(i) providing a nucleic acid molecule comprising a hairpin with a single-stranded loop and a double-stranded stem containing a target site for the nucleic acid ligase and/or the nucleic acid nuclease, wherein the nucleic acid molecule has a first end tethered to a surface and a second end remote from the first end, and wherein a detectable label is attached to the nucleic acid molecule either at the second end or between the target site and the second end;
(ii) contacting the nucleic acid molecule with the nucleic acid ligase or the nucleic acid nuclease; and
(iii) detecting the presence or absence of the detectable label, thereby determining activity of the nucleic acid ligase or the nucleic acid nuclease.

The method may further comprise the steps of denaturing and re-annealing the hairpin after step (ii) and prior to or simultaneously with step (iii).

The method may further comprise a washing step after step (ii) and prior to or simultaneously with step (iii).

In one embodiment, the method determines activity of the nucleic acid ligase. Here, the target site in the stem of the hairpin preferably comprises a single strand nick. The nick may be repaired in the presence of the nucleic acid ligase in step (ii) of the present method, thereby allowing detection of the presence of the detectable label in step (iii) of the present method to be correlated with nucleic acid ligase activity.

In another embodiment, the method determines activity of the nucleic acid nuclease. Here, the target site in the stem of the hairpin preferably comprises a nucleic acid nuclease cleavage site. In one embodiment, a single strand nick is formed at the nucleic acid cleavage site in the presence of the nucleic acid nuclease in step (ii) of the present method, thereby allowing detection of the absence of the detectable label in step (iii) of the present method to be correlated with nucleic acid nuclease activity. In an alternative embodiment, a double strand break is formed at the nucleic acid cleavage site in the presence of the nucleic acid nuclease in step (ii) of the present method, thereby allowing detection of the absence of the detectable label in step (iii) of the present method to be correlated with nucleic acid nuclease activity.

The stem of the hairpin may consist of 12 to 36, preferably 12 to 26, nucleotide pairs. For example, the stem of the hairpin may consist of 20 nucleotide pairs, with 6 to 12 nucleotide pairs located between the target site and the detectable label. As another example, the stem of the hairpin may consist of 26 nucleotide pairs, with 6 to 12 nucleotide pairs located between the target site and the detectable label.

The first end of the nucleic acid molecule is tethered to the surface. For example, the first end of the nucleic acid molecule may be tethered to the surface using a streptavidin-biotin link, a gold-thiol link, or a gold-thiolate link.

The term “label” as used herein refers to any compound attached or attachable to a nucleotide or nucleotide polymer, wherein the attachment may be covalent or non-covalent. The label is detectable and renders the nucleotide or nucleotide polymer detectable to the practitioner of the invention. Thus, the label may be a luminescent molecule, phosphorescent molecule, chemiluminescent molecule, fluorophore, coloured molecule, redox active molecule (such as a ferrocene group), radioisotope or scintillant. Most preferably the label is a fluorophore. The term “probe” commonly used in the art is for all intents and purposes of this invention equivalent to the term “label”.

The method for detecting the label may be selected for example from the group consisting of fluorescence, absorbance, electrochemistry, fluorescence resonance energy transfer (“FRET”), luminescence resonance energy transfer (“LRET”), fluorescence cross-correlation spectroscopy (“FCCS”), flow cytometry, direct quenching, ground-state complex formation, chemiluminescence energy transfer (“CRET”), bioluminescence energy transfer (“BRET”) and excimer formation.

The nucleic acid molecule of the present invention may be a DNA molecule or an RNA molecule or a DNA/RNA hybrid molecule.

The nucleic acid ligase of the invention may be a DNA ligase such as a prokaryotic DNA ligase (NAD⁺-dependent or ATP-dependent) or a eukaryotic ATP-dependent DNA ligase.

In a further embodiment, the nucleic acid ligase of the invention may be an RNA ligase. For example, the RNA ligase may be one of those produced by bacteriophage T4 which catalyse ligation of a 5′ phosphoryl-terminated nucleic acid donor to a 3′ hydroxyl-terminated nucleic acid acceptor through the formation of a 3′→5′ phosphodiester bond, with hydrolysis of ATP to AMP and PPᵢ. The substrates of the T4 RNA ligases include single-stranded RNA, single-stranded DNA and double-stranded molecules consisting of RNA alone or DNA/RNA hybrids.

The nucleic acid nuclease of the invention may alternatively be a DNA nuclease. In a preferred embodiment, the DNA nuclease is an endonuclease such as a restriction endonuclease. As used herein, a “restriction endonuclease” (also known as a “restriction enzyme”) means an enzyme which cuts (i.e. nicks, breaks or cleaves the phosphodiester backbone of) a DNA molecule at or near a specific site nucleotide sequence.

Restriction endonucleases are classified into type I, type II and type III. Of these, type II restriction endonucleases are preferred according to the present invention as they cut DNA at defined positions close to or within their recognition sequences. The most common type II enzymes are those like Hha I, Hind III and Not I that cleave DNA within their recognition sequences (for example, 5′-GCG↓C-3′, 5′-A↓AGCTT-3′ and 5′-GC↓GGCCGC-3′ for Hha I, Hind III and Not I, respectively, where “↓” indicates the cleavage site in the 5′ to 3′ direction). Most type II restriction endonucleases recognise DNA sequences that are symmetric because they bind to DNA as homodimers, but a few restriction endonucleases (e.g. BbvC I: 5′-CC↓TCAGC-3′), recognize asymmetric DNA sequences because they bind as heterodimers.

When restriction endonucleases bind to their recognition sequences in DNA, they usually hydrolyse both strands of the double-stranded duplex at the same time. Two independent hydrolytic reactions proceed in parallel, driven by the presence of two catalytic sites within each enzyme, one for hydrolysing each strand. Restriction enzymes that hydrolyse only one strand of the duplex, to produce DNA molecules that are “nicked” rather than cleaved, are referred to as “nicking endonucleases” and are preferred according to one aspect of the present invention. Nicking endonucleases available from New England BioLabs (Beverly, Mass., USA) include N.BstNB I (5′-GAGTCNNNN↓N-3′), N.Alw I (5′-GGATCNNNN↓N-3′), N.BbvC IA (5′-GC↓TGAGG-3′) N.BbvC IB (5′-CC↓TCAGC-3′) and Nb.Bsm I (3′-CTTAC↓GN-5′; note strand orientation). The preferred nicking endonucleases N.BbvC IA and N.BbvC IB, derivatives of the heterodimeric restriction enzyme BbvC I, are each engineered to possess only one functioning catalytic site and thus nick within the recognition sequence but on opposite strands.

According to a further aspect of the invention, the target site of the hairpin corresponds to or encompasses a restriction endonuclease site. A specific hairpin may thus be used to determine the activity of (or determine the quantity and/or quality of) a specific restriction endonuclease. In some cases, where restriction endonucleases are isoschizomers (i.e. where the enzymes recognise the same restriction site), a hairpin with a target site encompassing a restriction endonuclease site may be used to determine the activity of (or determine the quantity and/or quality of) one or more isoschizomer restriction endonucleases.

The invention in another aspect utilises a nicked oligonucleotide hairpin substrate immobilised via one terminus or end to a surface, and a detectable (for example, fluorophore) label on the remote terminus. The immobilised substrate is exposed to a ligase then denatured to disrupt base-pairing in the stem of the hairpin. If the substrate has been ligated by the ligase, the label is retained by a continuous covalent link of the hairpin, but the label is lost if ligation does not occur. In a reverse procedure, the activity of a nuclease can be detected by providing an intact oligonucleotide hairpin substrate as above which becomes nicked (i.e. cleaved in a single strand) or broken (i.e. cleaved in both strands) in the presence of the nuclease. Following denaturation, the label is retained in the absence of nuclease activity, but the label is lost if nuclease activity has disrupted the continuous covalent link of the hairpin.

The method of the invention encompasses detecting the presence or absence of a nucleic acid ligase or a nucleic acid nuclease by determining whether or not any nucleic acid ligase or nucleic acid nuclease activity is present in a sample.

Bacterial and viral enzymes are also routinely used in molecular biology assays and reagent suppliers test the activity of the enzymes before sale. The present method has advantages over the prior art methods in providing a rapid and accurate assay for ligases and nucleases.

The term “surface” includes any of the group consisting a bead, plate (for example, microtitre plate or a multiwell plate such as a 96-well or 364-well microtitre plate), a microarray, an electrode (for example, a metal electrode surface or a carbon electrode surface), glass and quartz.

In the present application, the stem of the hairpin is double-stranded due to pairing between purine and pyrimidine bases in adjacent sequences of the nucleic acid that are complementary.

According to a further aspect of the invention there is provided a method of determining whether a substance is a modulator of a nucleic acid ligase or a nucleic acid nuclease, comprising the steps of determining activity of the nucleic acid ligase or the nucleic acid nuclease using the method as described above in the presence and absence of the substance, thereby determining whether the substance is a modulator of the nucleic acid ligase or the nucleic acid nuclease. The modulator may, for example, be an antiseptic, antimicrobial, antibacterial, or antiviral agent.

According to another aspect of the invention there is provided a method of determining the quantity and/or quality of a nucleic acid ligase or a nucleic acid nuclease, comprising the steps of determining activity of the nucleic acid ligase or the nucleic acid nuclease using the method as described above in the presence of a known amount of the nucleic acid molecule, thereby determining the quantity and/or quality of the nucleic acid ligase or the nucleic acid nuclease.

Also provided according to the present invention is a nucleic acid molecule comprising a hairpin with a single-stranded loop and a double-stranded stem containing a target site for a nucleic acid ligase and/or a nucleic acid nuclease, wherein the nucleic acid molecule has a first end tethered to a surface and a second end remote from the first end, and wherein a detectable label is attached to the nucleic acid molecule either at the second end or between the target site and the second end. Features of the nucleic acid molecule may be as described above. The stem of the hairpin in a preferred embodiment consists of more than 5 base pairs to allow interaction with a nucleic acid ligase and/or a nucleic acid nuclease.

According to another aspect of the invention there is provided a kit comprising the nucleic acid molecule as defined above.

Further provided according to the invention is a method of determining activity of a nucleic acid repair moiety, the method comprising the steps of:
(i) providing a nucleic acid molecule comprising a hairpin with a single-stranded loop and a double-stranded stem containing a predetermined site of defined DNA damage at a target site for a nucleic acid nuclease, wherein the nucleic acid molecule has a first end tethered to a surface and a second end remote from the first end, and wherein a detectable label is attached to the nucleic acid molecule either at the second end or between the target site and the second end;
(ii) contacting the nucleic acid molecule with the nucleic acid repair moiety, whereby any repair of DNA damage by the repair moiety will create a target sequence at the target site, which target sequence is the target of the nuclease;
(iii) contacting the nucleic acid molecule with the nucleic acid nuclease;
(iv) detecting the presence or absence of the detectable label, thereby determining activity of the nucleic acid nuclease; and

(v) correlating the nuclease activity to repair capacity activity of the repair moiety.

### BRIEF DESCRIPTION OF THE DRAWINGS

So that the manner in which the above-recited features, advantages and objects of the invention, as well as others which will become apparent, are attained and can be understood in detail, more particular description of the invention briefly summarized above may be had by reference to the embodiments thereof which are illustrated in the drawings, which drawings form a part of this specification. It is to be noted, however, that the appended drawings illustrate only preferred embodiments of the invention and are therefore not to be considered limiting of its scope as the invention may admit to other equally effective embodiments.

In the drawings:

FIG. 1 is a diagram showing the use of tethered, nicked DNA hairpins for the voltammetric detection of DNA ligase activity.

FIG. 2 provides gels showing ligation of nicked DNA hairpins by the NAD⁺-dependent DNA ligase from E. coli.

FIG. 3 provides voltammograms showing the influence of E. coli NAD⁺-dependent DNA ligase on cyclic voltammetry from gold electrodes coated with ferrocene-terminated nicked hairpins.

FIG. 4 is a histogram showing quantification of the influence of E. coli DNA ligase on cyclic voltammetric peak areas displayed by ferrocene-terminated hairpins tethered to gold electrodes.

FIG. 5 provides gels showing ligation of solutions of nicked DNA hairpins by E. coli DNA ligase (LigA) as characterised by denaturing gel electrophoresis. Each hairpin has 3′ biotin and 5′ fluorescein, and the oligonucleotides are visualised by fluorescence of the latter.

FIG. 6 shows fluorescence data of a representative example of in-well ligation of immobilised nicked DNA hairpins by E. coli DNA ligase (LigA).

FIG. 7 is a graph showing normalised fluorescence retention for in-well ligation of immobilised nicked DNA hairpins as a function of E. coli DNA ligase (LigA) and T4 DNA ligase concentration.

FIG. 8 shows the results of an experiment assessing the effect of quinacrine—an inhibitor of NAD+-dependent DNA ligases—on the extent of ligation by E. coli DNA ligase (LigA).

FIG. 9 shows the results of an experiment using in-well ligation to follow purification of His-tagged E. coli DNA ligase (LigA) from a cell extract.

FIG. 10 shows the results of an experiment using in-well ligation from one sample of E. coli DNA ligase (LigA) transferred from well-to-well.

FIG. 11 shows the results of an experiment using in-well initiation of ligation from one sample of E. coli DNA ligase (LigA).

FIG. 12 shows the results of experiments assessing in-well ligation by DNA ligase and RNA ligases from bacteriophage T4.

FIG. 13 is a diagram showing the use of a biotin-streptavidin tethered DNA hairpin with a fluorescein (F) label for assaying nucleases that break DNA, RNA or DNA/RNA hybrids.

FIG. 14 provides gels showing nicking of solutions of DNA hairpins by N.BbvCIA as characterised by denaturing gel electrophoresis. Each hairpin has 3′ biotin and 5′ fluorescein, and the oligonucleotides are visualised by fluorescence of the latter. Oligonucleotides 1, 2 and 3 are as indicated in Table 4.

FIG. 15 shows the results of an experiment using in-well nuclease activity (N.BbvCI.IA).

FIG. 16 shows the results of an in-well experiment to assess the activity of commercially-available restriction endonucleases. Part (A) highlights where the restriction endonucleases cleave the hairpin.

FIG. 17 shows the concept of nicking and re-ligating a tethered DNA hairpin.

FIG. 18 shows the results of an experiment using in-well nuclease activity (N.BbvCI.IA) followed by in-well ligation (LigA) to follow sequential processing of a single DNA hairpin.

FIG. 19: Use of sequential nuclease and ligase activities to assess base excision repair (BER). (A) Schematic diagram of the proposed assay for BER of a damaged base, indicated by X. Box=restriction site, FL=fluorescein, B=biotin, S=streptavidin. (B) Interpretation of results from assays performed in part (A), with indication of their meaning in terms of BER.

### DETAILED DESCRIPTION OF THE PREFERRED EMBODIMENTS

Illustrative preferred embodiments of the invention are described below. In the interest of clarity, not all features of an actual implementation are described in this specification. It will nevertheless be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### EXAMPLE 1

### Tethered DNA Hairpins Facilitate Electrochemical Detection of DNA Ligation

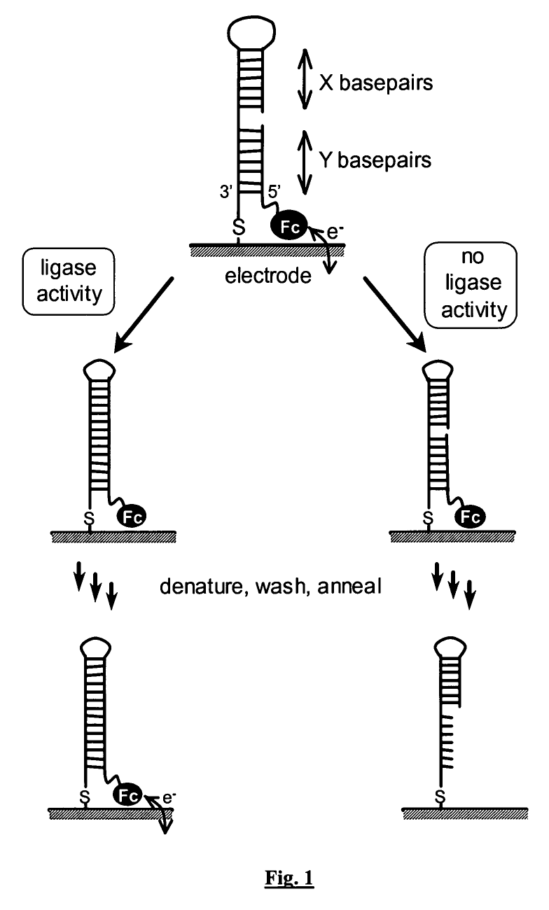

In a first example, the invention employs a nicked DNA hairpin as the ligase substrate, as shown in FIG. 1. The hairpin is tethered to a gold electrode through a terminal thiolate. A ferrocene label at the remote terminus provides a redox reporter for rapid characterisation of DNA status by cyclic voltammetry. Successful ligation of the DNA substrate is indicated by retention of the ferrocene couple after incubation with DNA.

Experimental Protocols

Materials

Expression and purification of the NAD⁺-dependent DNA ligase, LigA, from Escherichia coli was as described previously (Wilkinson et al., 2003, Proteins: Structure, Function & Genetics 51: 321-326; Lavesa-Curto et al., 2004, Microbiol. 140: 4171-4180). Oligonucleotides A-J (see Table 1) were supplied by MWG Biotech or SIGMA-Genosys. All other reagents were of Analar quality or equivalent and water was of resistivity >18 MΩ cm (Elga PureLab Maxima).

Preparation of Ferrocene-Terminated Oligonucleotide

Synthesis of ferrocene-terminated oligonucleotide, Fc-E, from the amine-terminated oligonucleotide, E, and ferrocene carboxylic acid N-succinimide ester (Molecular Sensing) was based on the method of Ihara and coworkers (Ihara et al., 1996, Nucleic Acids Res. 24: 4273-4280). Crude purification of oligonucleotide product was by gel-filtration with a Pharmacia NAP-10 column (0.1 M triethylammonium acetate, pH 6.8). The void volume was subject to reverse-phase HPLC with a Luna 5μ C18(2) column (150×4.6 mm) employing mobile phases of 0.1 M triethylammonium acetate, pH 6.8 and 10% acetonitrile (10 to 30% acetonitrile applied over 20 minutes at 1 mL min⁻¹). Elution of Fc-E at 13 minutes was detected by an increase of absorbance at 260 nm. Purified Fc-E was concentrated and exchanged into 20 mM Hepes, 1 M NaCl, pH 7.0. Fc-E concentration was estimated using ε_{260 nm}=135 700 M⁻¹cm⁻¹ calculated from the values of 15 400, 11 500, 8 700 and 7 400 M⁻¹cm⁻¹ for the A, G, T and C respectively and 9 500 M⁻¹cm⁻¹ for the ferrocene label. Control experiments performed with D showed no evidence of a reaction with ferrocene carboxylic acid N-succinimide ester and confirmed the amine terminus as the site of ferrocene addition to E.

Preparation of Nicked DNA Hairpins

Nicked hairpins carrying an identical six base loop but with variable stem lengths and positioning of the nick within the stem were prepared by hybridisation of pairs of partially complementary oligonucleotides. The resultant hairpins were distinguished by the nomenclature ‘X+Y’ where the nick is positioned X basepairs from the loop and Y basepairs from the foot of the hairpin, as shown in FIG. 1. In FIG. 1, nicked hairpins are identified with the ‘X+Y’ nomenclature that relates the position of the nick to the number of basepairs in the stem of the hairpin as indicated in the upper panel. Thus, hybridisation of oligonucleotides A and B (Table 1) forms ‘8+6’, A and C forms ‘14+6’, E (or F) and G forms ‘8+12’, E (or F) and H (or J) forms ‘14+12’. Oligonucleotides were dissolved in 90 mM Tris-borate, 10 mM EDTA, pH 8.3 (TBE) buffer. Those (B, C, G, H and J) defining the 5′ side of the nick were phosphorylated by T4 polynucleotide kinase (AbGene), purified by ethanol precipitation and resuspended in TBE buffer. Nicked DNA hairpins were formed with equimolar concentrations (typically 19 μM) of the appropriate oligonucleotides, heated at 90-100° C. for 5 minutes and slow-cooled to room temperature.

Characterisation of DNA Hairpins in Solution

Nicked DNA hairpins (50 pmoles) were incubated with DNA ligase (20 pmoles) in 10 μL of 26 μM NAD⁺, 10 mM MgCl₂, 25 μg mL⁻¹ bovine serum albumin, 10 mM dithiothreitol, 50 mM Tris-HCl, pH 8 at 25° C. for 1 hour. Control experiments were performed under identical conditions without the addition of DNA ligase. For product analysis samples were subjected to non-denaturing electrophoresis (15% polyacrylamide gel, 80 V, 6 hours, TBE buffer) or combined with an equal volume of formamide loading buffer, heated to 95° C. and subjected to denaturing electrophoresis (15% polyacrylamide-urea gel, 300 V, 1 hour, TBE buffer). Reaction products were visualised and quantitated using a Molecular Dynamics Storm phosphorimager.

Preparation and Voltammetric Characterisation of Tethered DNA Hairpins

Gold electrodes of ca. 4 mm diameter were prepared on glass microscope slides by vacuum evaporation of ˜20 nm chromium followed by 180 nm gold. Immediately prior to use electrodes were cleaned with warm (60-70° C.) piranha solution (70% concentrated sulfuric acid, 30% peroxide solution (30%)) for 30 min (CAUTION: PIRANHA SOLUTION MAY REACT VIOLENTLY WITH ORGANICS), rinsed thoroughly with water and dried with a flow of N₂ gas. Typically 1 μL of 20 μM hairpin, 20 mM Hepes, 1 M NaCl, pH 7.0 was placed on an electrode and left in a humidified chamber at room temperature for 3 to 16 hours as desired. The electrode was rinsed thoroughly with water then 20 mM Hepes, 1 M NaCl, pH 7.0. Non-specific interactions between the thiolated DNA and the gold surface were removed by exposure to 1 M mercaptoethanol for 2 hours (Herne & Tarlov, 1997, J. Am. Chem. Soc. 119: 8916-8920). Finally, electrodes were rinsed with 1 M NaClO₄, 25 mM Na₂HPO₄/NaH₂PO₄, pH 7.0 and stored in this buffer until use, typically within 2 days of preparation. Ligations were performed with 2.6 μM DNA ligase in 37 μM NAD⁺, 5.7 mM MgCl₂, 0.15 mM mercaptoethanol, 7 mM Hepes, pH 7.5 for 90 or 160 minutes at 37° C. Denaturation of the immobilised hairpins with 0.5% sodium dodecyl sulfate, 0.5 M NaOH was at room temperature.

Electrochemical measurements were performed with a three-electrode cell configuration housed in a N₂-filled chamber (atmospheric O₂<2 ppm). A KCl saturated Ag/AgCl reference electrode contacted the cell through a Luggin tip and a platinum wire formed the counter electrode. Voltammetry was performed at 23° C. with an Autolab 30 potentiostat under the control of GPES software. Potentials are reported relative to SHE by addition of 197 mV to that measured.

Results

Selection of a Nicked DNA Hairpin

Successful implementation of the present ligase assay is dependent on a number of factors. For good signal intensity, the nicked hairpins are preferably the predominant species on the electrode, i.e., the dissociation constant describing separation of its two strands must be low. This can be readily achieved with ‘long’ oligonucleotides but is preferably balanced by a desire to keep the hairpin short for economic reasons. The hairpin should also display the nick in such a way that it is accessible to the ligase. The dimensions of DNA ligases suggest that the electrode surface and hairpin loop should preferably be separated by at least 60 Å for successful ligation in the hairpin stem (Lee et al. 2000, EMBO J. 19: 1119-1129). To maximise the chance that the immobilised hairpins would stand proud of the surface a mercaptohexyl linker would be positioned at the 3′ terminus (Sam et al., 2001, Langmuir 17: 5727-5730).

With these points in mind the properties of four nicked hairpins were screened using the standard assay for activity of the NAD⁺-dependent ligase from E. coli (LigA), as described above. Product characterisation in this solution phase assay was by gel electrophoresis so the nicked hairpins carried a 5′ fluorescein label for visualisation. A stem of fourteen base pairs (ca. 48 Å) combined with a fully extended mercaptohexyl linker (ca. 11 Å) was considered to be the minimum length that could support ligation. Therefore, hairpins with stems of 14, 20 and 26 base pairs were designed in which the position of the nick relative to the loop was varied. These hairpins are referred to by an ‘X+Y’ nomenclature where X represents the number of base pairs between the loop and the nick, and Y represents the number of base pairs between the nick and the foot of the stem (see FIG. 1).

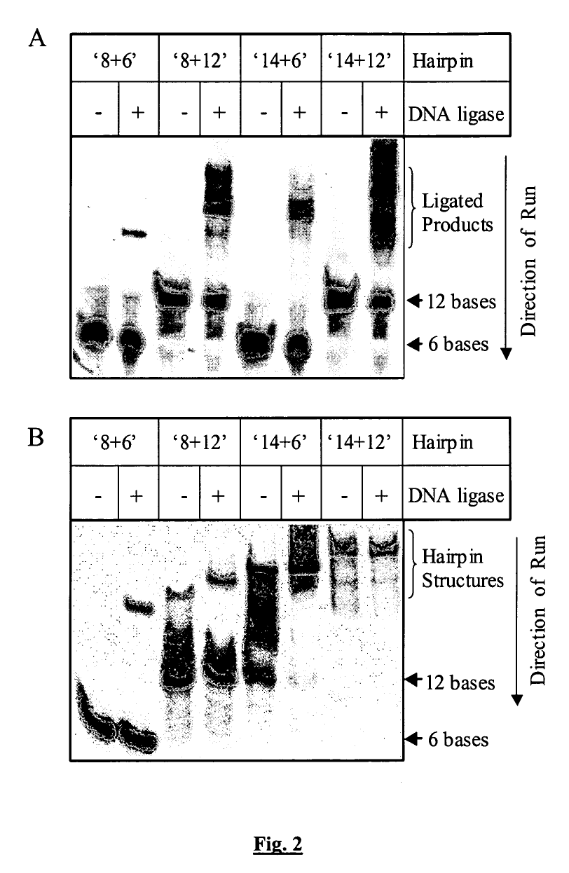

The extent of ligation achieved in each hairpin was assessed by electrophoresis under denaturing conditions on a 15% polyacrylamide gel, as shown in FIG. 2A. In FIGS. 2A and B, in vitro incubations using the indicated DNA substrate were performed without or with DNA ligase (lanes shown as “−” and “+”, respectively). Arrows indicate the 6- and 12-base oligonucleotides containing the 5′-fluorescein from the unligated, nicked hairpins. Successful ligation is indicated by greater retardation of the longer fluorescein labelled DNA strands that are present in the samples. Quantitation of the extent of ligation showed that the ‘8+12’ and ‘14+12’ hairpins were more effectively ligated, 49 and 41% respectively, than those hairpins with six basepairs between the nick and the foot of the hairpin, i.e., ‘8+6’ (6%) and ‘14+6’ (35%).

Further analysis of the suitability of the nicked hairpins for the desired assay through assessment of their structural integrity was provided by gel electrophoresis under non-denaturing conditions on a 15% non-denaturing polyacrylamide gel, as shown in FIG. 2B. A high affinity between the oligonucleotides forming the nicked hairpins will produce a high proportion of hybridised oligonucleotides, which will have different electrophoretic mobility compared to non-hybridised DNA. Furthermore, the mobility of this single band should be unaffected by ligation. This is seen for the ‘14+12’ sample. By contrast the ‘8+12’ and ‘8+6’ samples show a much greater population of fluorescein labelled single strand, especially for substrates not exposed to DNA ligase. The presence of some single-stranded fluorescein-terminated oligonucleotide is suggested by smearing in the ‘14+6’ sample prior to ligase exposure.

Due to the propensity of the ‘14+12’ sample to form nicked hairpins that are amenable to ligation this substrate was selected for exploration of the feasibility of ligating tethered nicked hairpins. The 3′ mercaptohexyl linker was omitted from hairpins used in the initial screening of nicked hairpin behaviour. Control experiments established that ‘14+12’ hairpins differing only in the presence or absence of the 3′ linker showed indistinguishable ligation efficiencies in assays comparable to those described above (data not shown).

Ligation of Tethered, Nicked DNA Hairpins.

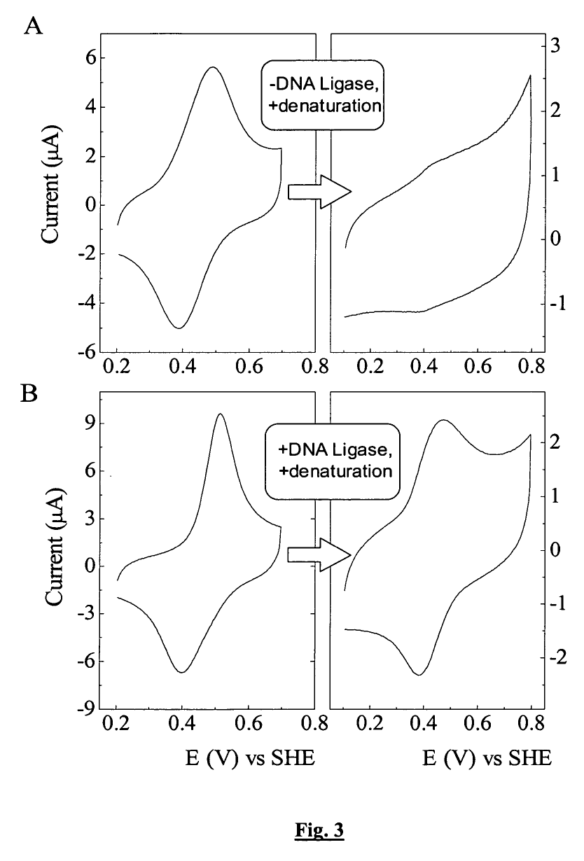

Gold electrodes coated with 3′ mercaptohexyl terminated ‘14+12’ bearing 5′ ferrocene gave cyclic voltammograms containing a pair of peaks, as shown in FIG. 3. In FIG. 3A, voltammograms were recorded before and after exposure to denaturation buffer and washing to remove non-covalently bound material. In FIG. 3B, voltammograms were recorded before and after exposure to DNA ligase, denaturation buffer and washing as described for FIG. 3A. Cyclic voltammetry was measured in 0.1 M NaClO₄ at a scan rate of 0.1 Vs⁻¹; other details are as described in the Experimental Protocols above. The peaks in FIG. 3 could be attributed to the oxidation and reduction of ferrocene since they were absent when experiments were performed with oligonucleotides lacking a ferrocene label. The peak areas are similar for the oxidative and reductive processes indicating reversibility of the electrode reaction. Integration of the peaks and the assumption of a flat electrode yields coverages on the order of 1×10¹⁴ molecules cm⁻². Given that the maximum theoretical density of hydrated duplex DNA is ca. 3×10¹³ molecules cm⁻² the observed value is most likely to reflect roughness of the electrode surface. Under these conditions interactions between neighboring molecules may account for deviation of the peak shapes from those predicted for identical, independent redox systems and that are observed from less densely packed assemblies of similar molecules. Indeed the relative distortion of the wavepair in FIG. 3B as compared to FIG. 3A may reflect the higher electroactive coverage of the electrode in the former experiment.

Exposure of hairpin coated electrodes to conditions that denature the duplex stem followed by rinsing to remove non-covalently bound material resulted in loss of >98% of the ferrocene response, as shown in FIG. 3A. Signals were lost after 5 minutes exposure to the denaturation conditions and longer incubations produced no further change to the voltammetric response. In contrast, a clear ferrocene signal was retained by electrodes exposed to ligase prior to exposure to denaturing conditions, as shown in FIG. 3B. These peaks were retained essentially unaltered after a further 10 and 30 minutes exposure to denaturation conditions. Thus, DNA ligase was able to perform covalent attachment of ferrocene to the electrode through ligation of the tethered nicked hairpin substrate.

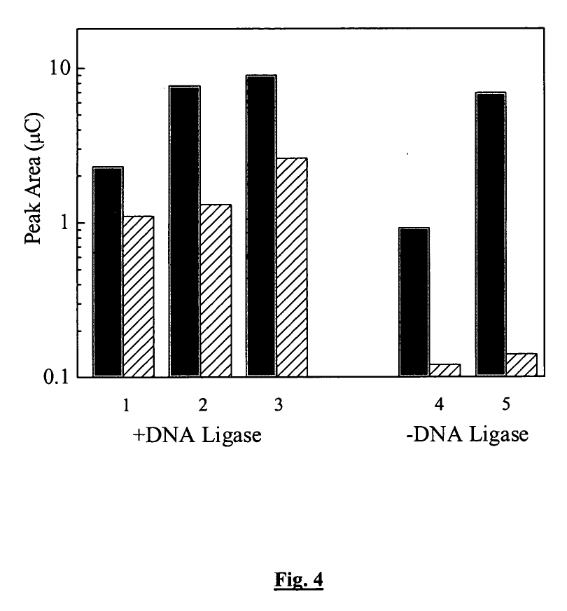

The voltammetric peak areas from several experiments with hairpin-coated electrodes are presented in FIG. 4. In FIG. 4, solid bars show initial peak areas displayed by five, independently prepared electrodes coated with ferrocene terminated nicked hairpins. The striped bars show peak areas after exposure to ligase reaction buffer that included or omitted DNA ligase, exposure to denaturation buffer and washing to remove non-covalently bound material. The approximately 10-fold variation of the initial coverage of the electrodes was achieved through variation of the time allowed for hairpin adsorption. None of the electrodes exposed to DNA ligase retained 100% of their initial signal intensity. This is in line with the results from electrophoretic analysis (see FIG. 2A). However, the number of molecules retained after ligation was remarkably similar in all experiments at ca. 5×10¹³ molecules cm⁻² (leading to an apparent ligation efficiency between 10 and 50% across the electrodes studied as illustrated in FIG. 4). This suggests that a constant population of tethered molecules is accessible to ligase perhaps due to roughness of the surface that renders some nicks inaccessible to the active site of the enzyme. An alternative explanation that the dense substrate packing may prevent access to certain populations of the tethered substrate seems less likely as this would result in lower populations of ligated product at electrodes with higher surface coverage, which was not observed.

Discussion

In Example 1 we have shown a novel electrochemical assay of DNA ligase activity. The enzyme used in our studies, E. coli LigA, has served as the paradigm for elucidation of the properties of NAD⁺-dependent DNA ligases, a family of proteins of molecular weight ca. 74 kDa and extensive amino acid sequence homology (Wilkinson et al., 2001, Molec. Microbiol. 40: 1241-1248). The smaller ATP-dependent DNA ligases (MW ca. 30-50 kDa) also form a homologous group and we have found that the enzyme from bacteriophage T4 also readily ligates the ‘14+12’ hairpin. Thus, this hairpin provides a suitable substrate for assaying DNA ligases from many different organisms. This generality of substrate for DNA ligases of different types is in large part due to the relative insensitivity of DNA ligase action to the sequence context of the nick (Alexander et al., 2003, Nucleic Acids Res. 31: 3208-3216). This property contrasts to that of many other DNA processing enzymes. However, DNA hairpins designed to contain appropriate DNA sequences should allow analysis of a number of DNA processing enzymes through an approach similar to that described here. For example, the activity of nucleases that introduce nicks into duplex DNA could be detected by reversing the assay described.

In terms of DNA ligase analysis the electrochemical methodology described here produces results in good qualitative agreement with those from the traditional electrophoretic analysis. Importantly, the simplicity and success of the voltammetric end-point assay demonstrates that DNA ligase action can be studied in proximity to a gold surface. This provides a framework from which necessarily more complex methods can be confidently pursued for resolution of enzyme action in real-time. When conditions for 100% ligation of the tethered hairpins have been defined, methods such as in situ scanning probe microscopies, surface plasmon resonance and attenuated total internal reflection-Fourier transform infrared spectroscopies should provide such information with resolution at the molecular and sub-molecular levels (see for example Wegner et al., 2003, Anal. Chem. 75: 4740-4746; Argaman et al., 1997, Nucleic Acids Res. 25: 4379-4384; and Bryson et al., 2000, Eur. J. Biochem. 267: 1390-1396). It may also be possible to exploit the time-base of the electrochemical analysis to monitor specifically changes in the conformation and/or rigidity of the DNA duplex during ligation. This could be done by positioning the redox reporter on the side of the nick remote from the electrode to allow the rates of electron conduction through the π-stack of the DNA duplex to be measured (Boon et al., 2002, Nature Biotech. 20: 282-286).

### EXAMPLE 2

### Tethered DNA Hairpins Facilitate Fluorescent Detection of DNA Ligation

In a preferred embodiment of the invention exemplified in Example 2, a nicked DNA hairpin tethered to a solid support at one terminus and with a covalently linked fluorophore at the remote terminus provides a means for rapid end-point detection of DNA ligase activity. The concept is as illustrated in FIG. 1, except that the DNA hairpin can be tethered to a solid support other than electrodes and a fluorophore rather than a ferrocene group is linked to the remote terminus of the hairpin. Immobilisation of nicked hairpins on streptavidin coated surfaces is afforded preferably by a 3′-biotin label. Assessment of hairpin status is afforded preferably by a 5′-fluorescein label. The assay facilitates high-throughput screening of DNA ligation efficiency, for example when performed in multi-well (micro-titre) plates. Since the traditional and widely used electrophoretic analysis of DNA ligation does not readily lend itself to high-through put analysis, this novel screening approach will be useful for the timely identification of ligation inhibitors that represent potential drug candidates.

Materials and Methods

Expression and purification of the NAD⁺-dependent DNA ligase from Escherichia coli (LigA) was as described in Example 1. The ATP-dependent DNA ligase from T4 was expressed and purified by a similar strategy. Oligonucleotides, Table 2, were supplied by MWG Biotech or SIGMA-GenoSys. Biotinylated strands were dissolved in aqueous solution, ethanol precipitated and resuspended to 100 μM in 50 μg/mL bovine serum albumin, 1 mM dithiothrietol, 4 mM MgCl₂, 30 mM Tris-HCl, pH 8.1. Fluorescein-labelled oligonucleotides were dissolved to 100 μM in 50 μg/mL bovine serum albumin, 1 mM dithiothrietol, 4 mM MgCl₂, 30 mM Tris-HCl, pH 8.1. The biotinylated strand was phosphorylated during synthesis by MWG Biotech/SIGMS-GenoSys or using standard enzymatic procedures (Sambrook et al., 2001, “Molecular Cloning: A Laboratory Manual”, 3rd edition ed., Cold Spring Harbor Laboratory Press, US).

Nicked DNA hairpins were prepared by mixing the appropriate biotinylated and fluorescein-labelled oligonucleotides (5:7 vol:vol, respectively), heating to 90° C. followed by gradual cooling to room temperature. Complete hairpins were formed in 5 or 100 μM solutions of the appropriate oligonucleotide, heated to 90° C. followed by gradual cooling to room temperature. The behaviour of immobilised oligonucleotides prepared from either complete hairpins was noted when immobilised from solutions of these two stock concentrations indicating that there is little intermolecular hybridisation.

Ligation of solutions of nicked hairpins was performed with 36 pmol LigA and 420 pmol nicked hairpin in 10 μL of Ligation Buffer (26 μM NAD⁺, 50 μg/mL bovine serum albumin, 1 mM dithiothrietol, 4 mM MgCl₂, 30 mM Tris-HCl, pH 8.1). The reaction was incubated at 37° C. for 90 minutes. For analysis the sample was combined with an equal volume of formamide loading buffer, heated to 95° C. and subjected to denaturing electrophoresis on a 15% polyacrylamide-urea gel at 300 V for 45 minutes. Experiments with T4 DNA ligase utilised similar enzyme concentrations to those use for LigA but the NAD⁺ of the Ligation Buffer was replaced by 1 mM ATP. Control experiments were performed in an identical way without DNA ligase.

Ligation of immobilised nicked hairpins was performed in the streptavidin coated wells of a micro-titre plate (StreptaWell HighBind 96-well plate, opaque, white). After each of the following steps were performed, the entire solution in the well was removed and stored or discarded, as required. Hairpins were immobilised by placing 80 μL of a 5 μM hairpin solution into each well. The hairpin solution was removed after 5 minutes and the wells washed 5 times with 100 μL Wash Buffer (30 mM Tris-HCl, 4 mM MgCl₂, pH 8.1), which allowed a stable fluorescence signal to be obtained from the immobilised hairpins. For ligation reactions, 100 μL of the desired concentration of DNA ligase in the appropriate Ligation Buffer was introduced into the well and left for 30 minutes. To denature the double-stranded DNA, the wells were exposed to 100 μL Denaturation Solution (0.1 M NaOH, 0.5% sodium dodecyl sulphate) for 4 minutes. Washes with 100 μL Wash Buffer were performed to monitor hairpin status at appropriate points in the assay. All procedures were performed at room temperature unless stated otherwise and control experiments were performed in the absence of DNA ligase. Fluorescence was recorded with a FL×800 Microplate Fluorescence Reader (Bio-Tek Instruments Inc). Excitation filter 485 nm and emission filter 516 nm (both with a full half- width maximum of 20 nm).

Results

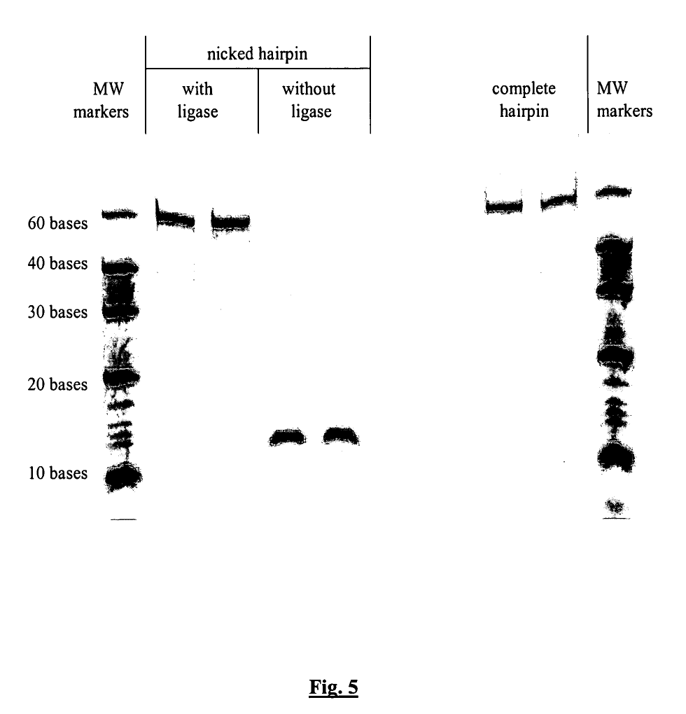

A biotin label on the 3′ terminus of the ‘14+12’ hairpin (see analogous FIG. 1) did not affect ligation of the hairpin by LigA when assessed by denaturing electrophoresis, and migration of the product was indistinguishable from that of the predicted ‘complete’ hairpin product that had been chemically synthesised, as shown in FIG. 5.

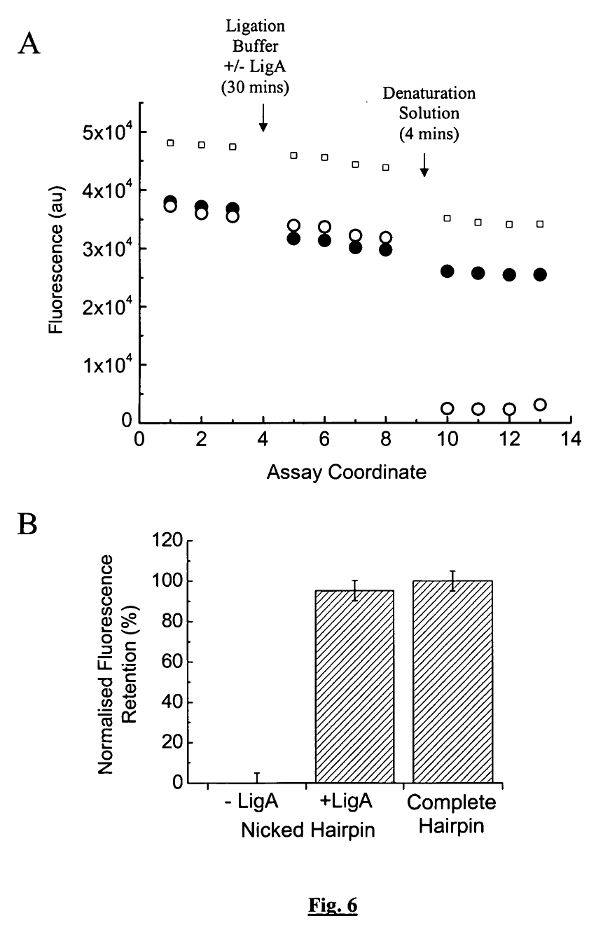

In-well ligation of the nicked DNA hairpins by LigA was reflected by retention of fluorescence after exposure to DNA ligase and denaturation conditions, as shown in FIG. 6. FIG. 6A shows raw data from a micro-titre plate reader, with (●) representing nicked hairpin with 180 nM LigA added at step 4, (603 ) representing nicked hairpin without LigA in step 4, and (58 ) representing complete hairpin without LigA in step 4. The assay coordinate starts when excess hairpin was removed from the well. Steps 1 to 3, 5 to 8 and 10 to 13 relate to exchange of Wash Buffer in the wells. FIG. 6B shows processed data from micro-titre plate reader. The retention of fluorescence after exposure to DNA ligase and denaturation conditions is in contrast to the almost complete loss of fluorescence noted when the experiment was repeated without DNA ligase. Parallel experiments performed with the synthetically prepared ‘complete’ hairpin showed a reproducible loss of fluorescence during the assay. This most likely reflects loss of hairpins from the plate triggered by exposure to the different solutions required by the assay and in particular the relatively harsh conditions required for denaturation. The efficiency of in-well ligation was calculated in the following manner. The fluorescence retained in control experiments with the nicked hairpin was taken as a measure of background fluorescence intensity. Fluorescence intensities after denaturation and washing of the hairpins (assay step 13, FIG. 6A) and immediately prior to exposure to Ligation Buffer (assay step 3, FIG. 6A) were corrected for this background. The ratio of the background corrected fluorescence intensities then gave a measure of the absolute % fluorescence retention for a given experiment. This value was normalised against the absolute % fluorescence retention for the complete hairpin calculated in the same manner.

A comparison of the ligation efficiencies recorded for LigA with two batches of nicked hairpin substrate through electrophoresis and in-well ligation methodologies is given in Table 3. There is good agreement for both sets of hairpins. The variation between hairpin batches most likely reflects the extent to which each set of hairpins bear a 5′ phosphate at the nick since this group is essential for ligation. Similar variations in levels of ligation are observed in the electrophoretic analysis of different batches of hairpins (Wilkinson et al., 2003, supra; Lavesa-Curto et al., 2004, supra).

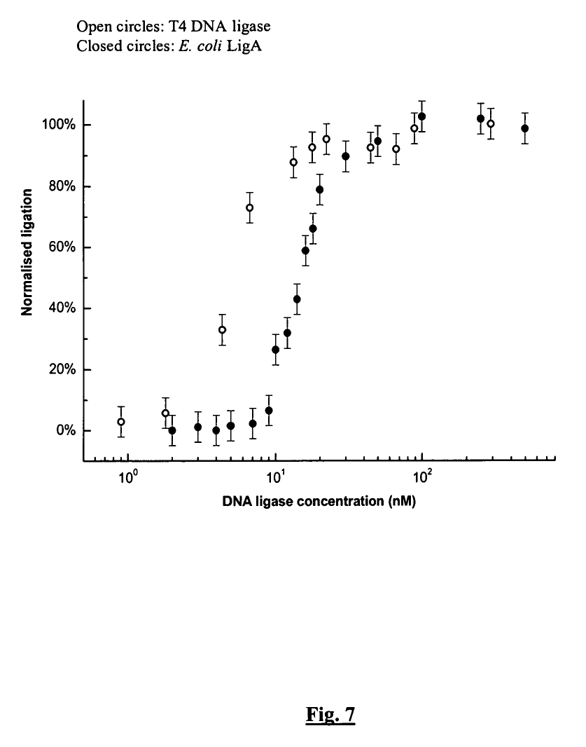

The extent of in-well ligation varies with enzyme concentration, as expected from knowledge of the activity of E. coli DNA ligase, is shown in FIG. 7. The in-well ligation of nicked hairpins was also accomplished by the ATP-dependent T4 DNA ligase, FIG. 7.

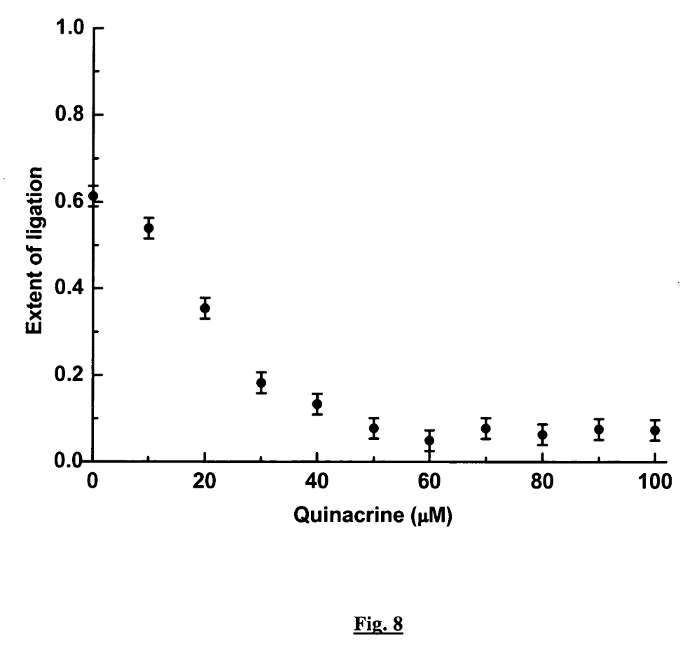

To confirm further that results for the proposed assay agree with those using established methods, we examined the activity of E. coli DNA ligase in the presence of quinacrine dihydrochloride, a known inhibitor of NAD⁺-dependent DNA ligases. To allow activation, or inhibition, of enzyme activity to be detected, this experiment used 15 nM of E. coli DNA ligase, a concentration that ligated 60% of immobilized hairpins under the reaction conditions without inhibitor. Our assay showed 50% inhibition of E. coli DNA ligase activity at approximately 20 μM quinacrine (FIG. 8). This confirms that the assay could be useful for the identification of inhibitors of the essential NAD⁺-dependent bacterial DNA ligases, which could produce promising targets for novel antibiotics.

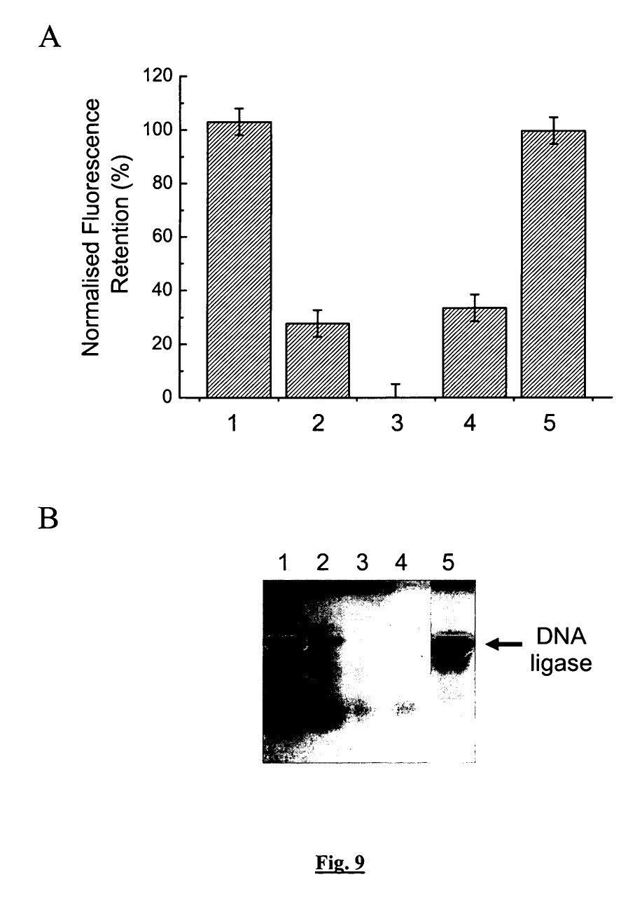

In addition to detecting ligation with purified enzyme, the assay works with cell extracts that contain a wide range of proteins, as shown in FIG. 9. FIG. 9A shows normalised fluorescence retention for in-well ligation of immobilised nicked DNA hairpins, while FIG. 9B shows Coomassie-stained SDS-PAGE. The columns are labelled as follows: (1) crude cell extract, (2) Ni-NTA column flow through, (3) 0.005 M imidazole wash of Ni-NTA column, (4) 0.06 M imidazole was of Ni-NTA column, and (5) 1 M imidazole wash of Ni-NTA column.

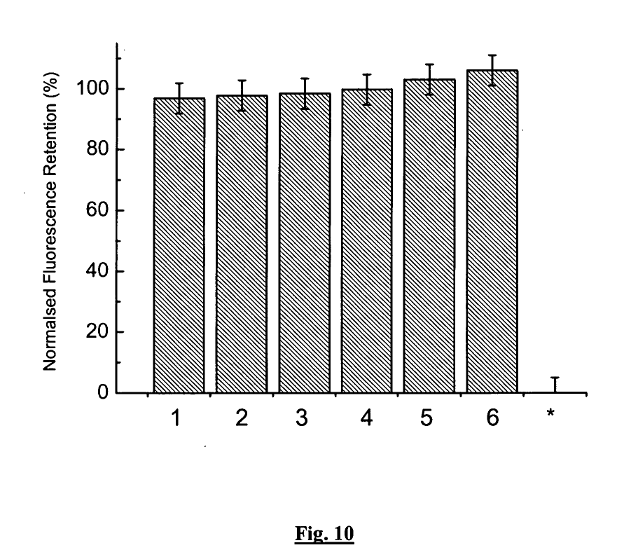

Immobilisation of the hairpin can be exploited to allow the reactivity of a ligase sample to be tested against various substrates simply by transfer of sample from well to well, as shown in FIG. 10. In FIG. 10, columns 1 to 6 show fluorescence retention for one sample of LigA transferred from well 1 to 6, while column * shows fluorescence retention in the absence of LigA.

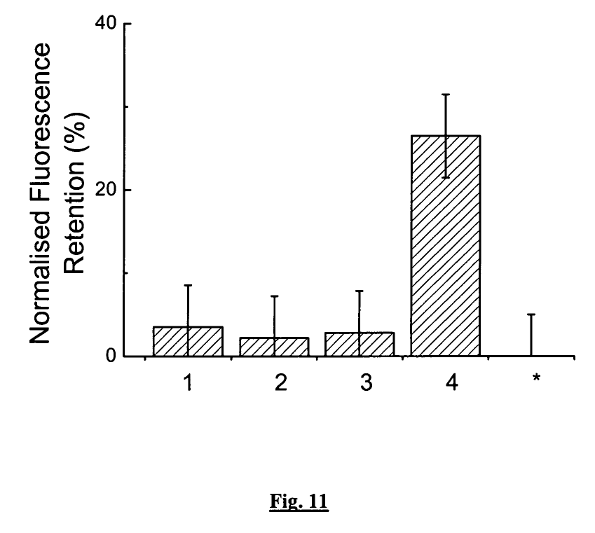

Biochemical details of the protein activity can be addressed by addition of cofactors to initiate reactions, for which see FIG. 11. In FIG. 11, columns 1 to 4 show fluorescence retention from one sample of LigA transferred from well 1 to 4. Wells 1 to 3 contained no NAD⁺. Well 4 contained 26 μM NAD⁺. Column * shows fluorescence retention in the absence of LigA.

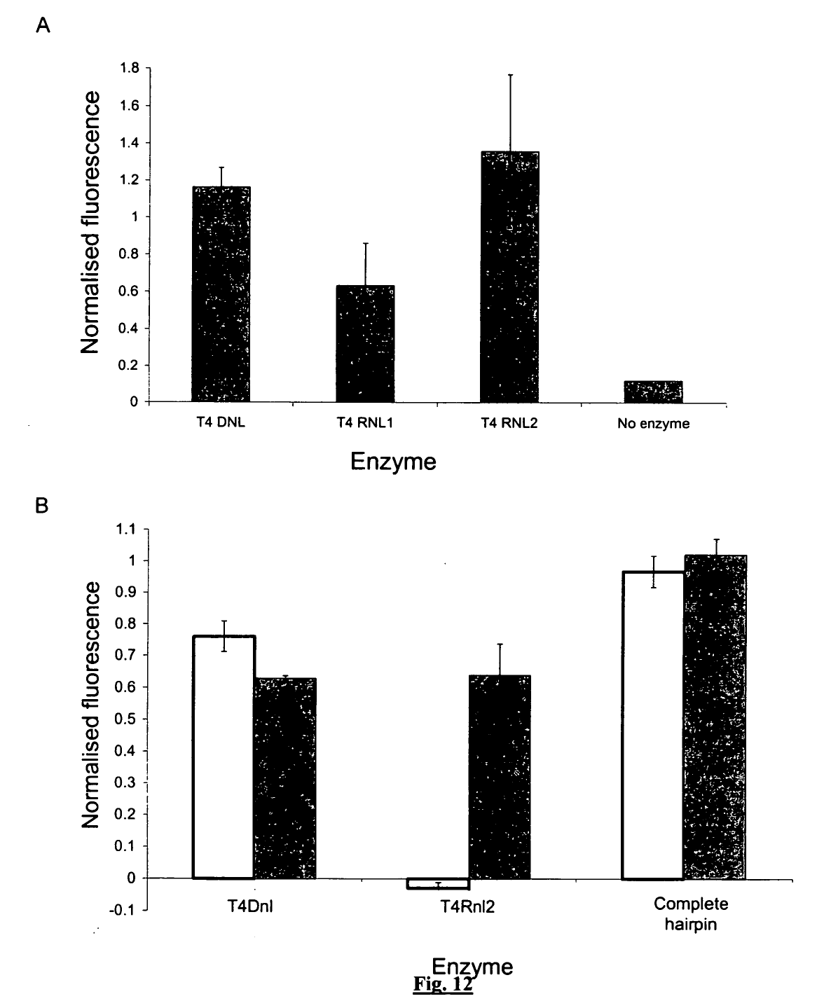

There is growing interest in understanding the metabolism of RNA. Furthermore, enzymatic ligation of RNA and RNA/DNA hybrids is useful in both commercial applications and for understanding the cellular roles of these proteins. The proposed assay can also be used to detect the biochemical activity of enzymes that ligate RNA/DNA hybrids (FIG. 12). An RNA/DNA hairpin was prepared with the biotinylated strand consisting of DNA and the fluorescein-labeled strand consisting of RNA (see FIG. 1 for comparison with a hairpin that consists entirely of DNA). Experiments with T4 DNA ligase and RNA ligases 1 and 2 from T4 confirms that the assay detects that all of these enzymes join nicks in RNA/DNA hairpins (gray bars in FIG. 12A). Additional incubations identify that T4 RNA ligase 2 cannot ligate hairpins consisting solely of DNA (white bars in FIG. 12B) and, so, require the presence of RNA in the hairpin to allow ligation activity to be detected.

### EXAMPLE 3

### Tethered DNA Hairpins Facilitate the Detection of nuclease Activity and the Sequential Nicking and Ligation of a Specific Piece of DNA

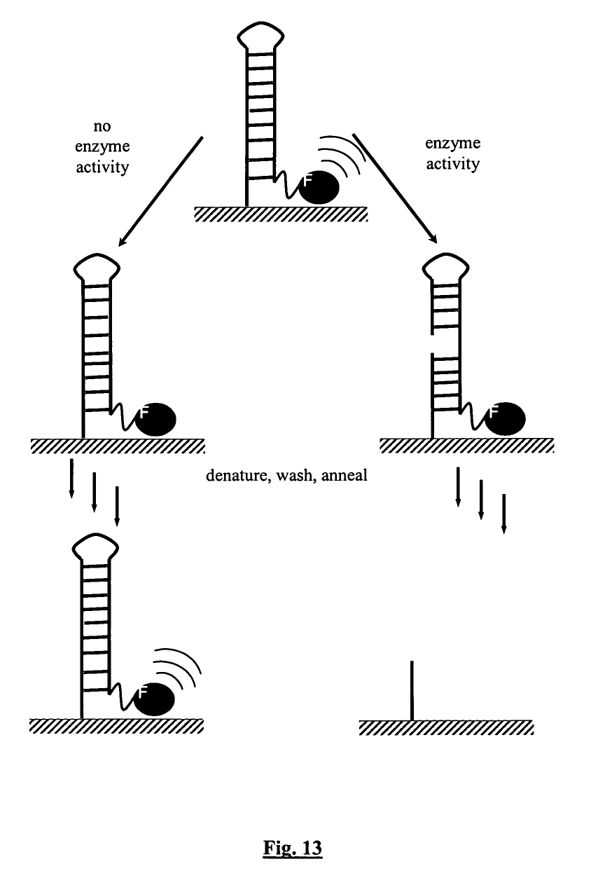

In a preferred embodiment of the invention exemplified in Example 3, a DNA hairpin tethered to a solid support at one terminus and with a covalently linked fluorophore at the remote terminus provides a means for rapid end-point detection of DNA nuclease activity. The tethered DNA hairpin contains a restriction site within the sequence of the stem and the assay concept is illustrated in FIG. 13. The assay is also used to demonstrate sequential nuclease and ligase activities on a specific, tethered DNA hairpin.

Materials and Methods.

Oligonucleotides are detailed in Table 4. Hybridisation of 2 and 3 formed a nicked hairpin that is the predicted product of N.BbvCI.IA action on 1. Oligonucleotides were resuspended in 10 mM Tris-HCl, pH 8.1 with 10 mM MgCl₂, purified by ethanol precipitation and resuspended in the same buffer. DNA ligases were obtained as in Examples 1 & 2. The nuclease N.BbvCIA was obtained from New England Biolabs. Nuclease assays were performed in Reaction Buffer B comprised of 10 mM Tris-HCl, pH 7.9, 50 mM NaCl, 10 mM MgCl₂ and 1 mM dithiothreitol.

In-well assays of ligase and nicking enzyme activity followed a common protocol and differed only in the “Reaction Step”. Each assay consisted of four steps:
1) Hairpins (80 μL of 5 μM hairpin in 10 mM Tris-HCl, pH 8.1 with 10 mM MgCl₂) were loaded into the wells and left for 5 minutes to saturate the available biotin-binding sites.
2) Wells were drained and washed five times with 100 μL Wash Buffer (30 mM Tris-HCl, pH 8.1, 4 mM MgCl₂) to remove free oligonucleotide and obtain a stable fluorescence from the immobilised hairpins.
3) The “Reaction Step” was as follows:
   For DNA Ligases:
Ligation Buffer (99 μL) containing 26 μM NAD⁺ or 1 mM ATP, as appropriate, was added to the wells followed by an aliquot (1 μL) of EcLigA or T4 DNA ligase. In control experiments 1 μL of 20 mM Tris-HCl, pH 7.5, 200 mM NaCl was introduced in place of the enzyme. Micro-titre plates were then left at room temperature for the desired reaction time, drained and exposed to 0.1M NaOH, 0.5% sodium dodecylsulphate as denaturant for 4 minutes. Experiments that explored the cofactor specificity of the ligases were performed as above except cofactor was omitted until desired.
   For Nicking by N.BbvCI.IA:
Reaction Buffer B (99 μL) was added to the wells followed by an aliquot (1 μL) of N.BbvCIA. In control experiments 1 μL of the enzyme storage buffer (Diluent A, New England Biolabs) was introduced in place of the enzyme. The wells were left at room temperature for the desired reaction time, drained and exposed to 0.1M NaOH, 0.5% sodium dodecylsulphate as denaturant for 4 minutes.
4) Wells were drained of denaturant and washed five times with 100 μL wash buffer to obtain a stable fluorescence reading.
   Results.

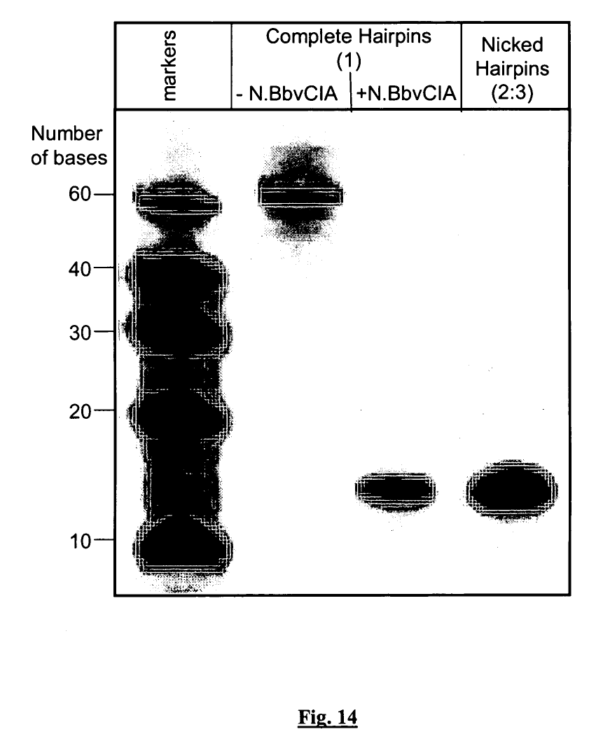

The structure of the hairpin did not affect the activity of N.BbvCI.IA when assessed by denaturing electrophoresis, and migration of the product was indistinguishable from that of the predicted ‘nicked’ hairpin product that had been chemically synthesised, as shown in FIG. 14.

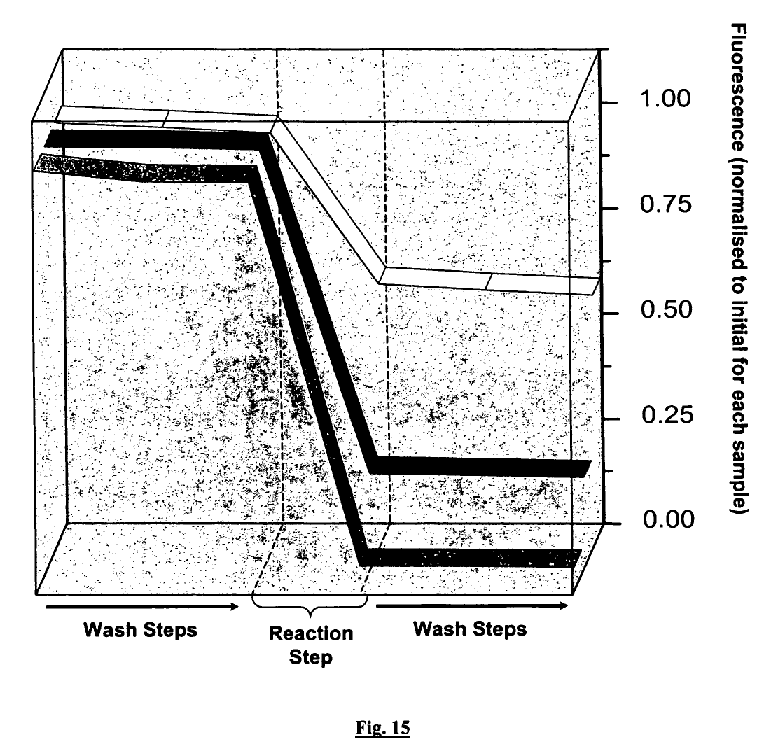

Results from a typical assay using tethered hairpins with N.BbvCI.IA are illustrated as part of FIG. 15. In FIG. 15, fluorescence retention has been normalised to that from hairpins containing the N.BbvCI.IA restriction site and exposed to parallel reaction conditions but lacking enzyme (white line, FIG. 15). Hairpins that were chemically-synthesised in a “nicked” format lost all fluorescence when exposed to the same reaction conditions (gray line, FIG. 15). A hairpin exposed to N.BbvCI.IA and denaturation conditions lost ˜70% fluorescence (black line, FIG. 15). Quantitative comparison of N.BbvCI.IA activity under conditions that led to complete nicking of the available hairpins confirmed that results achieved with immobilised hairpins and analysis by denaturing gel-electrophoresis were within experimental variation.

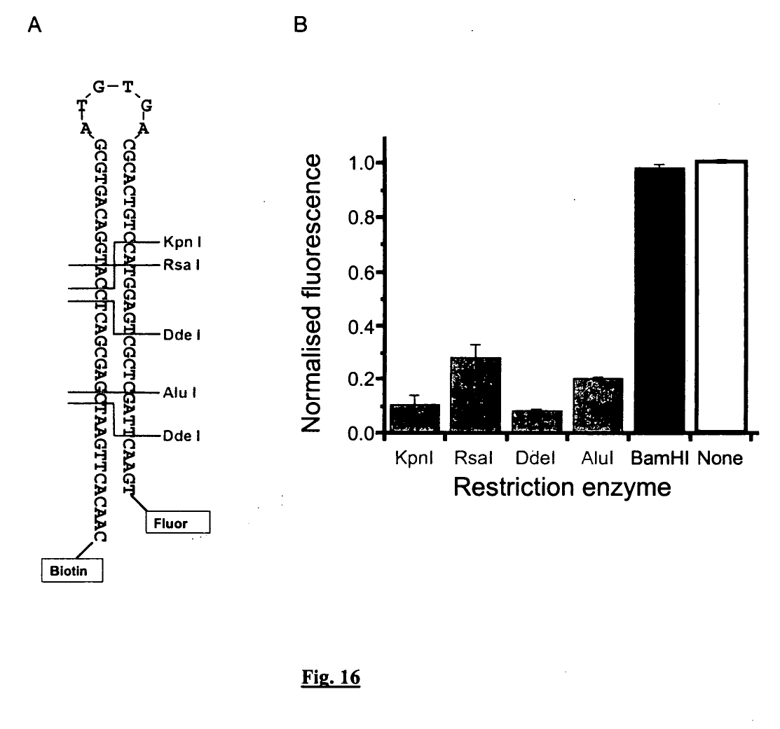

A wide variety of restriction endonucleases are produced by biotechnological and pharmaceutical companies and the proposed assay is suitable for determining their nuclease activity (FIG. 16). When incubated with several commercially-available restriction enzymes, the hairpins were cleaved if they contained the appropriate recognition of the added restriction enzymes (gray bars in FIG. 16), but not if the hairpin did not contain the recognition sequence (black bar in FIG. 16).

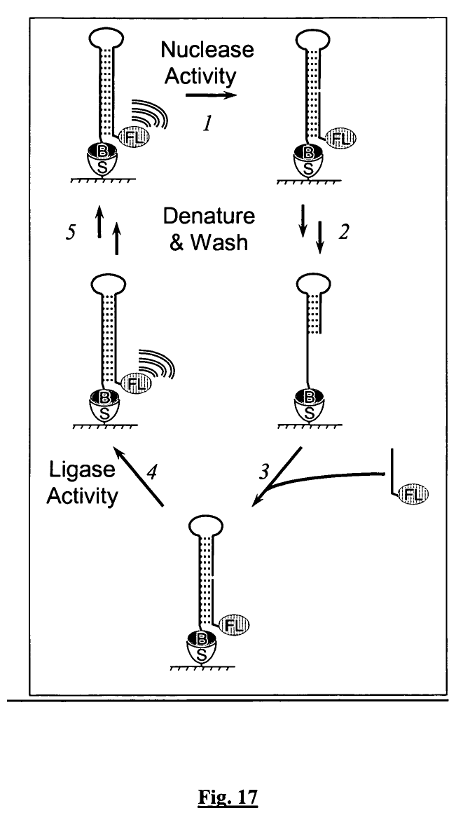

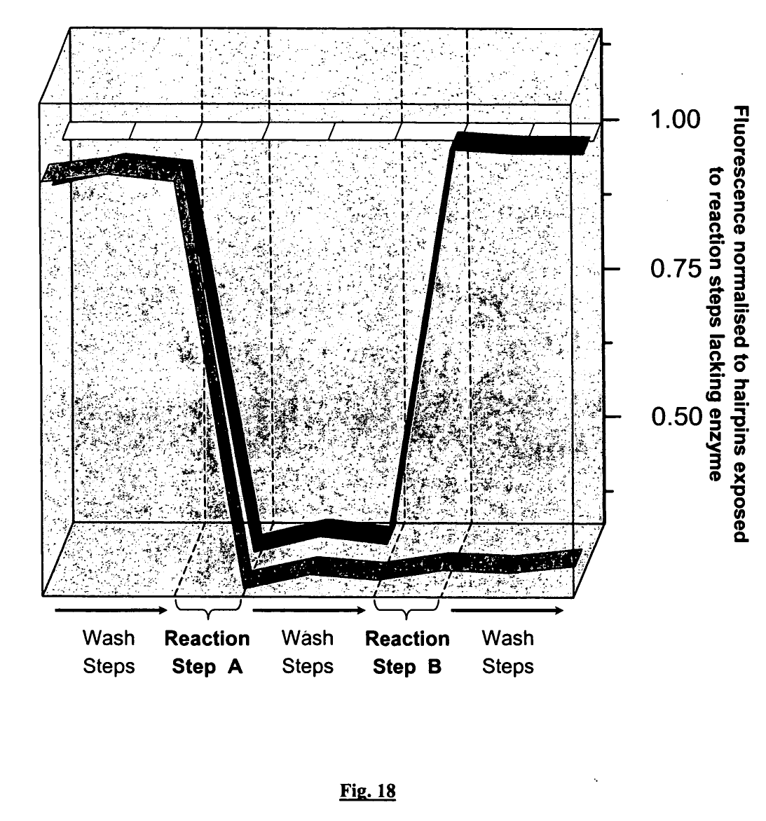

After the incubation with N.BbvCI.IA, the experimental protocol was then used to demonstrate that sequential reactions could be detected on immobilised hairpins. Accordingly, the products of the N.BbvCI.IA experiments were exposed to further reaction conditions as illustrated in FIG. 17. For analysis of the data (FIG. 18), fluorescence retention has been normalised to that from hairpins containing the N.BbvCI.IA restriction site and exposed to parallel reaction conditions but lacking enzyme (white line, FIG. 18). After incubation with N.BbvCI.IA in Reaction Step A, an excess of fluorescein-terminated oligonucleotide was added to all the samples to generate the predicted product of the N.BbvCI.IA reaction. Subsequent exposure to LigA and denaturation resulted in the fluorescence intensity (black line, FIG. 18) being restored to within error of that from the complete hairpin exposed to parallel conditions but without addition of enzymes (white line, FIG. 18). By contrast there was no recruitment of fluorescence when LigA was omitted from Reaction B (gray line, FIG. 18). Additional experiments confirmed that multiple cycles of nicking and ligation could be performed on single nucleic acid samples (data not shown).

### EXAMPLE 4

### Development of the Assay to Detect Repair of DNA Damage

It is estimated that up to 10,000 DNA damage events occur per human cell per day as a result of normal cellular function, in addition to exposure to environmental agents such the ultra-violet component of sunlight and cigarette smoke. The consequences of this DNA damage are diverse and include cell death and disease. In fact, DNA damage occurs with a frequency that is too high to be compatible with any form of life. Thus, maintaining the chemical integrity of DNA is an essential part of cellular function and every living system employs a number of mechanisms to detect and repair DNA damage. Characterising these repair mechanisms by existing methods is a time-consuming process.

The invention further provides a novel, robust approach to rapid detection of DNA repair. This method could relieve an existing bottle-neck to advancing the understanding of these essential processes. The assay uses fluorescently labelled DNA hairpins containing a site of defined DNA damage. These DNA hairpins will be immobilised on a surface (such as a plastic ‘plate’). This can facilitate running multiple experiments in a short time. Repair of the damage by any repair moiety (such as a biological sample) will generate a unique sequence of bases within the DNA. This sequence is the target of an enzyme (such as a restriction endonuclease) that cuts the backbone of DNA with the result that the fluorescently labelled repaired DNA is washed off the plate. By contrast, any remaining damaged DNA will remain bound to the plate along with its fluorescent label. Thus, quantitation of fluorescence loss provides an immediate read-out of DNA repair capacity by the repair moiety.

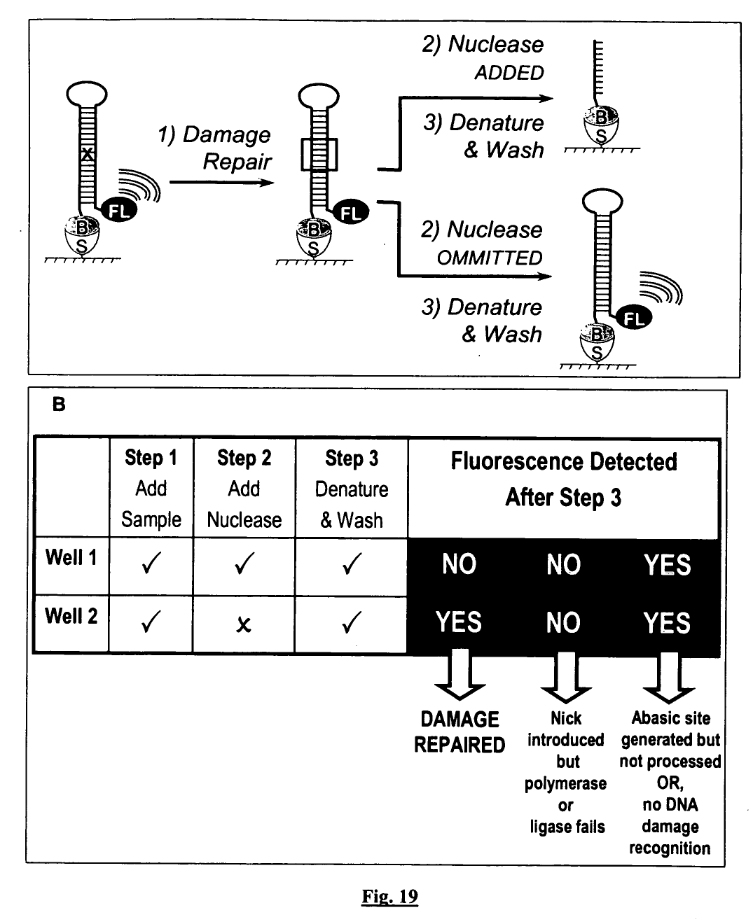

The DNA damage repair assay method is shown schematically in FIG. 19.

One use of this assay will be detection of base excision repair with purified enzymes. This will allow suitable sizes of DNA hairpin to be established for processing by protein complexes. Resistance of hairpins to non-specific degradation in cell extracts may be optimised as necessary. The length of hairpins can be extended to allow more complex mechanisms of damage repair to be assessed.

The damage repair assay will facilitate researchers engaged in elucidation of the fundamental elements of DNA damage repair and its capacity within the cellular context. Perhaps most significantly the assay provides opportunity for time-resolved quantitation of DNA damage repair capacities, which current experimental strategies make too laborious to contemplate. Results from such studies will contribute to the wider understanding of nucleic metabolism, construction of the E-cell and those engaged in developing pharmaceuticals. Some cancers are associated with polymorphisms in DNA damage repair proteins. Furthermore, some treatments for cancer produce damage that can be recognised, and reversed, by DNA damage repair proteins, which may reduce the efficacy of such therapies and even promote the proliferation of tumour cells that are resistant to treatment. The recent identification of small molecule inhibitors of DNA damage repair suggests pharmacological inhibition of repair mechanisms could be used to enhance the cytotoxicity of anticancer agents. Although such studies have generated much interest and excitement, additional optimisation of compounds is required and thus our assay and the results obtained with it will be of benefit to clinical and drug discovery markets.

Discussion

A novel assay of DNA ligase activity has been demonstrated. The assay has proved to be robust and it gives results in good agreement with those obtained by the traditional electrophoretic analysis when experiments are performed in parallel.

The novel ligation assay identifies ligation in relatively impure samples, and this may provide a useful quick approach to analyse DNA ligase expression in cells.

The ability to transfer protein samples from one well of the micro-titre plate to another without loss of activity allows protein samples to be assessed against a huge number of DNA targets. This is advantageous if the proteins are in short supply or expensive. Such analysis could not be performed easily with the traditional electrophoretic assay because it is not straightforward to separate the DNA and protein incubated together in solution.

In the present example, preparation and processing of the micro-titre plate during the novel assay was performed by manual pipeting. However, it should be relatively straightforward to automate the procedure to facilitate high-throughput analysis.

The present invention is also suitable for quantifying RNA ligation. There is increasing interest in enzymatic ligation of RNA and RNA/DNA hybrids both for use in commercial applications and for understanding the cellular roles of these proteins (see Shuman & Lima, 2004, Curr. Opin. Struct. Biol. 14: 757-764).

The present invention has been used to assay nuclease activity. There is considerable interest in the action of nucleases with DNA, RNA and RNA/DNA hybrids both for use in commercial applications and for understanding the cellular roles of these proteins.

The present invention has been used to investigate DNA processing. Ligation of DNA (and RNA) usually occurs as the final step in a sequential reaction. To have a full biochemical appreciation of how ligases and nucleases work it is important to see how their activities link with other proteins involved in the pathways. Coupling the approaches of the novel in-well ligation assay with exchange of protein sample (FIGS. 10 and 11) and the assay concept for nuclease activity (FIG. 13) has allowed such analyses to be performed carefully using purified proteins.

The foregoing examples are meant to illustrate the invention and do not limit it in any way. One of skill in the art will recognize modifications within the spirit and scope of the invention as indicated in the claims.

All references cited herein are hereby incorporated by reference.

TABLE 1

Summary of oligonucleotides used in Example 1.

| | |
|---|---|
| Oligonucleotide | Sequence |

| | | |
|---|---|---|
| A | 5′ F1-TGACTC 3′ | (SEQ ID NO:1) |
| B | 3′ ACTGAGCGAGTGCGAGTG | (SEQ ID NO:2) |
| | TACGCACTCG 5′ | |
| C | 3′ ACTGAGCGATGGACAGTG | (SEQ ID NO:3) |
| | CGAGTGTACGCACTGTCCATC | |
| | G 5′ | |
| D | 5′ TGAACTTAGCTC 3′ | (SEQ ID NO:4) |
| E | 5′ H₂N-TGAACTTAGCTC 3′ | (SEQ ID NO:5) |
| F | 5′ F1-TGAACTTAGCTC 3′ | (SEQ ID NO:6) |
| G | 3′ ACTTGAATCGAGCGAGTG | (SEQ ID NO:7) |
| | CGAGTGTACGCACTCG 5′ | |
| H | 3′ ACTTGAATCGAGCGATGG | (SEQ ID NO:8) |
| | ACAGTGCGAGTGTACGCACTG | |
| | TCCATCG 5′ | |
| J | 3′ RSS-ACTTGAATCGAGCG | (SEQ ID NO:9) |
| | ATGGACAGTGCGAGTGTACGC | |
| | ACTGTCCATCG 5′ | |

^{a}F1 = fluorescein label, RSS = disulfide label with (CH₂)₆ linker to oligonucleotide, H₂N = amino label with (CH₂)₆ linker to oligonucleotide. For oligonucleotides that can form a hairpin structure the bases of the loop are underlined.

TABLE 2

Summary of oligonucleotides used in Example 2.

| | |
|---|---|
| Olignucleotide | Sequence and Labels |

| | |
|---|---|
| ‘Complete’ | 5′ (fluorescein)-TGA ACT TAG CTC GCT |
| Hairpin | ACC TGT CAC GCA TGT GAG CGT GAC AGG |
| | TAG CGA GCT AAG TTC ACA AC-(biotin) |
| | 3′ |
| | (SEQ ID NO:10) |
| Nicked Hairpin | 5′ GCT ACC TGT CAC GCA TGT GAG CGT |
| Pt 1 | GAC AGG TAG CGA GCT AAG TTC ACA AC- |
| | (biotin) 3′ |
| | (SEQ ID NO:11) |
| Nicked Hairpin | 5′ (fluorescein)-TGA ACT TAG CTC 3′ |
| Pt 2 | (SEQ ID NO:6) |

TABLE 3
Comparison of ligation efficiencies in Example 2

| Experiment 1 | | Experiment 2 | |
|---|---|---|---|
| Solution ligation | | Solution ligation | |
| with | In-well ligation | with | In-well ligation of |
| electrophoretic | of immobilised | electrophoretic | immobilised |
| analysis | hairpins | analysis | hairpins |
| 100 ± 10% | 97 ± 5% | 47 ± 10% | 49 ± 5% |

TABLE 4

Oligonucleotide

| | |
|---|---|
| 1 | 3′ BTN-CAACACTTGAATCGAGCGACTCCATGGACAGTGCGAGTGTAC |
| | GCACTGTCCATGGAGTCGCTCGATTCAAGT-F1 |
| | SEQ ID NO:12 |
| 2 | 3′ BTN-CAACACTTGAATCGAGCGACTCCATGGACAGTGCGAGTGTAC |
| | GCACTGTCCATGGAGT-Pᵢ |
| | SEQ ID NO:13 |
| 3 | 5′ F1-TGAACTTAGCTCGC |
| | SEQ ID NO:13 |

BTN = biotin label, Pᵢ = 5′-phosphate, F1 = fluorescein label. Bases that form the loop in hairpin structures are underlined. Bases that define the N.BbvCI.IA recognition sequence are in bold.

What is claimed is

1. A method of determining activity of a nucleic acid ligase or a nucleic acid nuclease, the method comprising the steps of:
(i) providing a nucleic acid molecule comprising a hairpin with a single-stranded loop and a double-stranded stem containing a target site for the nucleic acid ligase and/or the nucleic acid nuclease, wherein the nucleic acid molecule has a first end tethered to a surface and a second end remote from the first end, and wherein a detectable label is attached to the nucleic acid molecule either at the second end or between the target site and the second end;
(ii) contacting the nucleic acid molecule with the nucleic acid ligase or the nucleic acid nuclease; and
(iii) detecting the presence or absence of the detectable label, thereby determining activity of the nucleic acid ligase or the nucleic acid nuclease.

2. The method of claim 1, further comprising the steps of denaturing and re-annealing the hairpin after step (ii) and prior to or simultaneously with step (iii).

3. The method of claim 1, further comprising a washing step after step (ii) and prior to or simultaneously with step (iii).

4. The method of claim 1, wherein the method determines activity of the nucleic acid ligase.

5. The method of claim 4, wherein the target site in the stem of the hairpin comprises a single strand nick.

6. The method of claim 5, wherein the nick is repaired in the presence of the nucleic acid ligase in step (ii) of claim 1, thereby allowing detection of the presence of the detectable label in step (iii) of claim 1 to be correlated with nucleic acid ligase activity.

7. The method of claim 1, wherein the method determines activity of the nucleic acid nuclease.

8. The method of claim 7, wherein the target site in the stem of the hairpin comprises a nucleic acid nuclease cleavage site.

9. The method of claim 8, wherein a single strand nick or a double strand break is formed at the nucleic acid cleavage site in the presence of the nucleic acid nuclease in step (ii) of claim 1, thereby allowing detection of the absence of the detectable label in step (iii) of claim 1 to be correlated with nucleic acid nuclease activity.

10. The method of claim 1, wherein the stem of the hairpin consists of 12 to 26 nucleotide pairs.

11. The method of claim 10, wherein the stem of the hairpin consists of 20 nucleotide pairs, with 6 to 12 nucleotide pairs located between the target site and the detectable label.

12. The method of claim 10, wherein the stem of the hairpin consists of 26 nucleotide pairs, with 6 to 12 nucleotide pairs located between the target site and the detectable label.

13. The method of claim 1, wherein the first end of the nucleic acid molecule is tethered to the surface using a streptavidin-biotin link, a gold-thiol link, or a gold-thiolate link.

14. The method of claim 1, wherein the detectable label is a fluorophore or a redox active molecule.

15. The method of claim 1, wherein the nucleic acid molecule is a DNA molecule or an RNA molecule or a DNA/RNA hybrid molecule.

16. The method of claim 1, wherein the nucleic acid ligase is a DNA ligase.

17. The method of claim 16, wherein the DNA ligase is a prokaryotic DNA ligase (NAD⁺-dependent or ATP-dependent) or a eukaryotic ATP-dependent DNA ligase.

18. The method of claim 1, wherein the nucleic acid ligase is an RNA ligase.

19. The method of claim 1, wherein the nucleic acid nuclease is a restriction endonuclease.

20. A method of determining whether a substance is a modulator of a nucleic acid ligase or a nucleic acid nuclease, comprising the steps of determining activity of the nucleic acid ligase or the nucleic acid nuclease using the method of claim 1 in the presence and absence of the substance, thereby determining whether the substance is a modulator of the nucleic acid ligase or the nucleic acid nuclease.

21. The method of claim 20, wherein the modulator is selected from the group consisting of an antiseptic agent; an antibacterial agent; an antimicrobial agent; and an antiviral agent.

22. A method of determining the quantity and/or quality of a nucleic acid ligase or a nucleic acid nuclease, comprising the steps of determining activity of the nucleic acid ligase or the nucleic acid nuclease using the method of claim 1 in the presence of a known amount of the nucleic acid molecule as defined in claim 1, thereby determining the quantity and/or quality of the nucleic acid ligase or the nucleic acid nuclease.

23. A nucleic acid molecule comprising a hairpin with a single-stranded loop and a double-stranded stem containing a target site for a nucleic acid ligase and/or a nucleic acid nuclease, wherein the nucleic acid molecule has a first end tethered to a surface and a second end remote from the first end, and wherein a detectable label is attached to the nucleic acid molecule either at the second end or between the target site and the second end.

24. The nucleic acid molecule of claim 23, wherein the target site in the stem of the hairpin comprises a single strand nick which is repairable by a nucleic acid ligase.

25. The nucleic acid molecule of claim 23, wherein the target site in the stem of the hairpin comprises a nucleic acid nuclease cleavage site, which site forms a single strand nick or a double strand break in the presence of a nucleic acid nuclease.

26. The nucleic acid molecule of claim 23, wherein the stem of the hairpin consists of 12 to 26 nucleotide pairs.

27. The nucleic acid molecule of claim 26, wherein the stem of the hairpin consists of 20 nucleotide pairs, with 6 to 12 nucleotide pairs located between the target site and the detectable label.

28. The nucleic acid molecule of claim 26, wherein the stem of the hairpin consists of 26 nucleotide pairs, with 6 to 12 nucleotide pairs located between the target site and the detectable label.

29. The nucleic acid molecule of claim 23, wherein the first end of the nucleic acid molecule is tethered to the surface using a streptavidin-biotin link, a gold-thiol link, or a gold-thiolate link.

30. The nucleic acid molecule of claim 23, wherein the detectable label is a fluorophore or a redox active molecule.

31. The nucleic acid molecule of claim 23, wherein the nucleic acid molecule is a DNA molecule or an RNA molecule or a DNA/RNA hybrid molecule.

32. A kit comprising the nucleic acid molecule of claim 23.

33. A method according to claim 1 comprising the step of:
(ii) contacting the nucleic acid molecule sequentially with the nucleic acid ligase and the nucleic acid nuclease.

34. A method according to claim 1 comprising the step of:
(ii) contacting the nucleic and molecule sequentially with the nucleic acid nuclease and the nucleic acid ligase.

35. A method according to claim 33 or 34 wherein sequential step (ii) is carried out in multiple cycles.

36. A method according to claim 33 comprising the step of:
(iii) determining the activity of both the nucleic acid ligase and the nucleic acid nuclease.

37. A method of determining activity of a nucleic acid repair moiety, the method comprising the steps of:
(i) providing a nucleic acid molecule comprising a hairpin with a single-stranded loop and a double-stranded stem containing a predetermined site of defined DNA damage at a target site for a nucleic acid nuclease, wherein the nucleic acid molecule has a first end tethered to a surface and a second end remote from the first end, and wherein a detectable label is attached to the nucleic acid molecule either at the second end or between the target site and the second end;
(ii) contacting the nucleic acid molecule with the nucleic acid repair moiety, whereby any repair of DNA damage by the repair moiety will create a target sequence at the target site, which target sequence is the target of the nuclease;
(iii) contacting the nucleic acid molecule with the nucleic acid nuclease;
(iv) detecting the presence or absence of the detectable label, thereby determining activity of the nucleic acid nuclease; and
(v) correlating the nuclease activity to repair capacity activity of the repair moiety.

38. The method of claim 37, wherein the defined DNA damage is modification of a base or bases within the DNA, and the repair capacity activity of the repair moiety is Base Excision Repair.

39. The method of claim 38 wherein Base Excision Repair of the nucleic acid molecule enables the nuclease to cut through the double-stranded stem of the nucleic acid molecule thereby releasing the detectable label from the tethered first end of the molecule.
